(19) United States
(12) Patent Application Publication
(10) Pub. No.: US 2017/0326305 A1
(43) Pub. Date: Nov. 16, 2017

(54) Needle Assembly with Needle Hub Shielding a Needle Cannula

(71) Applicant: Novo Nordisk A/S, Bagsvaerd (DK)

(72) Inventor: Jacqueline Trosborg, Kongens Lyngby (DK)

(21) Appl. No.: 15/527,377

(22) PCT Filed: Nov. 17, 2015
(86) PCT No.: PCT/EP2015/076776

§ 371 Date: May 17, 2017

(30) Foreign Application Priority Data
Dec. 1, 2014 (EP) 14195601.1

(51) Int. Cl. A61M 5/32; A61M 5/24

(52) U.S. Cl. CPC A61M 5/3202; A61M 5/3293; A61M 5/3221; A61M 2005/2474

(57) ABSTRACT

A needle assembly (1) comprising a needle unit (2), where the needle unit (2) comprises a hub (6) with a skirt (3) having a longitudinal central axis (A), where the skirt (3) is having a proximal (7) and a distal end (8). The skirt is also having an internal surface adapted to interface with a mounting portion of a drug delivery device. The needle unit further comprises a needle cannula (30) having a proximal (31) and a distal (32) end, a central hub portion (5) holding the needle cannula (30), a transition portion (4) attached to the skirt portion (3), and wherein the transition portion (4) connects the skirt (3) with the central hub portion (5). The needle unit (2) is adapted to be switched between a retracted mode and an ejected mode. In the retracted mode, the distal end (32) of the needle cannula (30) is in a position which is proximal to the distal end (8) of the skirt (3), and the needle cannula (30) may be safely accommodated within the skirt (3). In the ejected mode, the distal end (32) of the needle cannula (30) is in a position which is distal to the distal end (8) of the skirt (3), and the needle cannula (30) may be ready for insertion. The transition portion (4) is adapted to deform, and thereby enable the switching between the modes, by allowing the central hub portion (5) to be moved in a longitudinal direction.

### THE TECHNICAL FIELD OF THE INVENTION

The invention relates to a needle assembly to be mounted on a drug delivery device. More particularly, the invention relates to a needle assembly with a minimum of components, and with means for safe handling in a retracted mode.

### DESCRIPTION OF RELATED ART

Needle units for medical devices are used in large numbers and for various applications, where fluids are injected to or extracted from the body. Needle units can therefore interface with many different medical devices as syringes, pens, infusion sets etc.

Pen needle units are particularly used by people suffering from diabetes, who have to inject themselves with insulin at a daily basis. For this purpose a great number of different pen systems have been developed over the last 30 years. Common for pen injectors is that they contain a container or cartridge containing the liquid drug to be injected. In order to transfer the liquid drug from the injection pen and into the body of the patient a pen needle unit is used. Such double-pointed pen-needle units have a needle cannula with a non-injection end (proximal end) which penetrates into the cartridge when the pen needle unit is attached to the injection pen and a patient end (distal end) that enters into the body of the patient during injection to create a liquid communication between the inner of the cartridge and the patient. A pen needle unit further comprises a hub carrying the cannula and the hub is usually provided with means for attaching the pen needle assembly to the injection pen. However, pen needle units are also used to treat other medical diseases.

Double pointed needles for drug delivery devices are used in large numbers, and it is therefore desirable to improve production processes and the construction of needle assemblies in order to reduce the production costs.

Such needle assemblies for drug delivery devices are typically disposable and are discarded after one single use. The problem presented by the disposal of a needle assembly, and indeed, by any handling of the needle assembly, is the potential risk of being injured by any of the sharp ends of the needle cannula. This is particular dangerous when following after the penetration of a patients skin since the needle cannula then may be contaminated and therefore capable of spreading diseases such as hepatitis and HIV.

US 2003/0144633 A1 allegedly discloses a needle assembly comprising a double pointed needle, and a two-part needle support comprising an inner body slidingly accommodated in an outer body. The inner body is holding the needle cannula, and the needle assembly can be stored in an initial state.

### SUMMARY OF THE INVENTION

It is an object of the present invention to provide a needle assembly, where a needle unit can be contained in a sterile condition before use, and in a condition for safe disposal after use. Another object of the invention is to construct a needle assembly allowing reduced production costs, and where the needle unit can be sterilized in a bulk sterilization process. The invention may also solve further problems that will be apparent from the disclosure of the exemplary embodiments.

In one aspect, the present invention relates to a needle assembly comprising a needle unit, where the needle unit comprises:
a hub with a skirt or skirt portion having a longitudinal central axis, where the skirt is having a proximal and a distal end, and an internal surface adapted to interface with a mounting portion of a drug delivery device;
a needle cannula having a proximal and a distal end;
a central hub portion holding the needle cannula;
a transition portion attached to the skirt portion, and wherein the transition portion connects the skirt with the central hub portion;
wherein the needle unit is adapted to be switched between:
a retracted mode, where the distal end of the needle cannula is in a position which is proximal to the distal end of the skirt, and whereby the needle cannula may be safely accommodated within the skirt; and
an ejected mode, where the distal end of the needle cannula is in a position which is distal to the distal end of the skirt, and whereby the needle cannula may be ready for insertion; and wherein the transition portion is adapted to deform, and thereby enable the switching between the modes, by allowing the central hub portion to be moved in a longitudinal direction.

The present invention provides a needle assembly with a small number of parts, allowing safe handling of a needle before and after use. Due to the small number of parts this construction may allow reduced production costs, which are dependent on the number of parts. The present invention provides a needle assembly, where the needle cannula can be covered or shielded by the skirt of the needle hub, when the cannula is in the retracted state, and it may therefore not be necessary to provide any additional shields to minimize the risk of cannula related accidents. One function of the skirt may be providing an internal surface to interface with the needle mount of a drug delivery device. By additionally using the skirt as a needle shield, the needle assembly may provide a self-contained safety function, as it may not be required to use additional shields, i.e., the skirt provides an attachment and shieling function.

The needle unit can be switched between a retracted and an ejected mode. This functionality is enabled by the deformable or flexible transition portion, which connects the central cannula holding hub part with the skirt. In some embodiments the skirt, the transition portion and the central hub part may be a single unit, which may be injection moulded. In some embodiments, the needle cannula may also be injection moulded, and be an integral part of the former unit. In other embodiments the needle cannula, is a metal or polymer cannula otherwise connected to the hub.

The transition portion may be any deformable element which enables the function of switching the needle unit between the modes. In some embodiments the transition portion is adapted to change between a first shape in the retracted mode, and a second shape in the ejected mode. The deformation may be elastic, plastic or a combination thereof.

In some embodiments, a needle assembly according to the invention comprises a transition portion adapted to position the central hub portion in a first position, when the needle unit is in the retracted mode; and
wherein the transition portion is adapted to position the central hub portion in a second position, when the needle unit is in the ejected mode; and
wherein the first position is proximal to the second position.

The proximal end of the needle assembly is the non-injection end or non-patient end, and the distal end is the opposite end. Therefore, the central hub portion may be closer to the non-patient end in the first position than in the second position. As the central portion is holding the needle cannula, the needle cannula may also be positioned closer to the non-patient end in the first position than in the second position.

In some embodiments a needle assembly according to the present invention comprises a first sealing member. Furthermore, the skirt may have a first sealing support portion at the distal end of the skirt, wherein said first sealing support portion is adapted to support the first sealing element.

The needle units according to the present invention may be provided in an outer container sealed off by a sealing member. However, by applying the member directly to a sealing support at the distal end of the skirt, the distal end of the needle cannula may be completely covered. And the sealing member may at the same time enable bulk sterilization, as the sealing member may be permeable to sterilizing agents, and at the same time prevent diffusion off contaminants across the member. By eliminating the need of an outer cover, the present invention may provide a needle assembly with a reduced number of parts, and the construction may therefore allow reduced production costs.

In some embodiments, a needle assembly according to the present invention comprises a needle unit adapted to be reversibly switched between the retracted and the ejected mode, which means that the cannula may be returned after it has been advanced in the distal direction.

In some embodiments, a needle assembly according to the present invention comprises a needle unit adapted to be switched between:
an initial retracted mode, wherein the needle assembly is in the retracted mode, the first sealing element is supported by the first sealing support portion, and the distal end of the needle cannula and the first sealing element is separated by a minimum distance; and
the ejected mode.

The distal end of the needle cannula is initially covered by the first sealing member, and there is provided a distance between the distal end of the cannula and the sealing member, which is sufficient to prevent unintentional perforation of the sealing member during storage, transport and handling. The first time, the needle unit is switched from the retracted mode to the ejected mode, the sealing member must either be removed manually or it may be broken by the needle cannula, or some other suitable mechanism.

In some embodiments, a needle assembly according to the present invention comprises a second sealing support portion at the proximal end of the skirt, a second sealing element, and wherein the second sealing support portion is adapted to support the second sealing element. In some embodiments, the single sealing member, e.g. the first, may be adapted to be supported by the first and the second sealing support portions.

The proximal end of the needle cannula is thereby covered by a sealing member, and the needle assembly may be bulk sterilized through the first or the second sealing member. The sealing member covering the proximal end may be manually removed before the needle unit is mounted on the drug delivery device.

In some embodiments, a needle assembly according to the present invention comprises a second sealing element adapted to be broken by the drug delivery device, when the needle unit is mounted on the drug delivery device. The needle assembly can thereby be put directly onto a drug delivery device, whereby the number of steps in order to handle the needle assembly is reduced.

In some embodiments, a needle assembly according to the present invention comprises attachment means on the internal surface of the skirt, where the attachment means are adapted to attach the needle unit to the needle mount of the injection device. The attachment or connecting means could be a luer coupling, a bayonet coupling, a threaded coupling or any combination thereof e.g. a combination as described in EP 1,536,854.

In some embodiments, a needle assembly according to the present invention is adapted to be switched between the retracted and the ejected mode, when the needle unit is mounted on the drug delivery device. The needle assembly is put directly onto the drug delivery device, which may be an injection device, and during mounting the needle unit is automatically switched from the retracted to the ejected mode, whereby the first sealing element may be broken by the cannula. The injection device will thereafter be ready for insertion on ejection.

In some embodiments, a needle assembly according to the present invention comprises a central hub portion having a proximal surface adapted to interface with a structure of the drug delivery device, and wherein the needle unit is adapted to be switched between the retracted and the ejected mode, when a force having a longitudinal component is exerted on the proximal surface, wherein the central hub portion may be driven in a longitudinal direction during mounting of the drug delivery device. Thereby the drug delivery device may force the central hub portion in a distal direction during mounting.

In some embodiments, a needle assembly according to the present invention comprises a transition portion comprising a biasing member exerting a force on the central hub portion enabling automatic retraction from the ejected mode, when the drug delivery device is dismounted. The biasing member is thereby trying to restore the needle unit to the retracted mode. The biasing member may be obtain by providing a compressible transition portion, wherein the compressibility can be obtained by providing a compressible material, or by providing a compressible geometrical structure, e.g., a corrugated, spiral or spring like structure.

In some embodiments, a needle assembly according to the present invention comprises a transition portion comprising a bi-stable element, whereby the needle unit is adapted to be mechanically stable in the retracted and the ejected mode, and wherein the central hub portion, during the process of mounting the drug delivery device to the needle unit, may be switched between the retracted and the ejected mode through an intermediate mode, where the intermediate mode defines an unstable mode, where the central hub portion may flip to either of the two stable modes.

In another aspect, the present invention relates to a method of handling a needle assembly according to the present invention comprising:
providing the needle assembly; and
mounting the needle unit on the drug delivery device and thereby switching the needle unit from the retracted to the ejected mode.

In some embodiments, the present invention relates to a method of handling a needle assembly comprising the step of dismounting the needle unit from the drug delivery device and thereby switching the needle unit from an ejected to a retracted mode.

### BRIEF DESCRIPTION OF THE DRAWINGS

The invention will be explained more fully below in connection with detailed embodiment and with reference to the drawings in which:

FIG. 1 Shows a perspective view of a needle assembly according to the present invention with a first and a second sealing member

FIG. 2 Shows a cross section of the needle assembly in FIG. 1 and additional details of a distance between a cannula tip and a sealing member

FIG. 3 Shows a needle assembly according to an embodiment of the present invention being mounted on a drug delivery device with a needle unit in a retracted mode.

FIG. 4 Shows a needle assembly according to an embodiment of the present invention with a needle unit in an ejected mode, where a transition portion comprises a biasing member.

FIG. 5 Shows a needle assembly according to an embodiment of the present invention with a needle unit in an ejected mode, where a transition portion comprises a bi-stable element.

The figures are schematic and simplified for clarity, and they just show details, which are essential to the understanding of the invention, while other details are left out. Throughout, the same reference numerals may be used for identical or corresponding parts.

### DETAILED DESCRIPTION OF EMBODIMENT

When, in the following, terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as their relative dimensions are intended to serve illustrative purposes only.

In that context it may be convenient to define that the term “distal end” in the appended figures. As an example, when considering a needle cannula, the “distal end” is meant to refer to the end of the needle cannula penetrating the patient whereas the term “proximal end” is meant to refer to the opposite end pointing away from the patient in a situation of use.

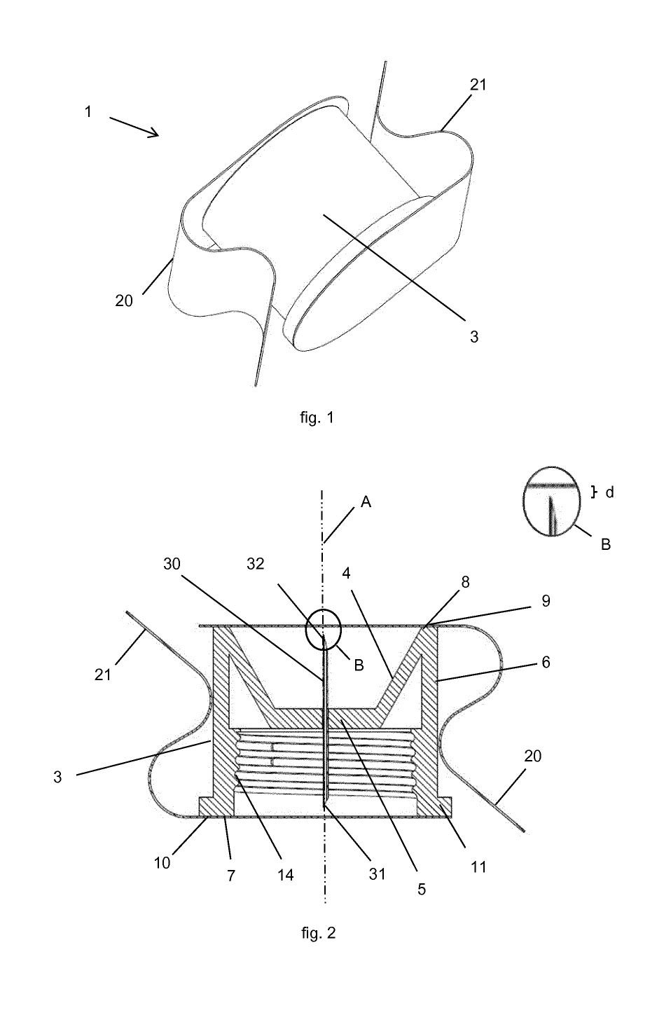

FIG. 1 shows a perspective view of needle unit 2, with a first 20 and a second sealing member 21. As shown on FIG. 2, the needle unit comprises a hub 6 with a skirt 3 having a longitudinal central axis (A). The skirt 3 is having a proximal end 7 and a distal end 8, and an internal surface adapted to interface with a mounting portion of a drug delivery device 40. In the embodiment of FIG. 2, the internal surface is provided with attachment means 14, which in this case is an internal thread corresponding to an external thread on a needle mount of a drug delivery device 40. However, the attachment or connecting means could also as examples be a luer coupling, a bayonet coupling or any combination thereof e.g. a combination as described in EP 1,536,854.

FIG. 2 also shows a needle cannula 30 having a proximal end 31 and a distal end 32, and a central hub portion 5 holding the needle cannula 30. The skirt 3 is connected to the central hub portion 5 through a transition portion 4 which is deformable or flexible, and, as explained below, the transition portion enables the transition or switching between different modes of the needle unit.

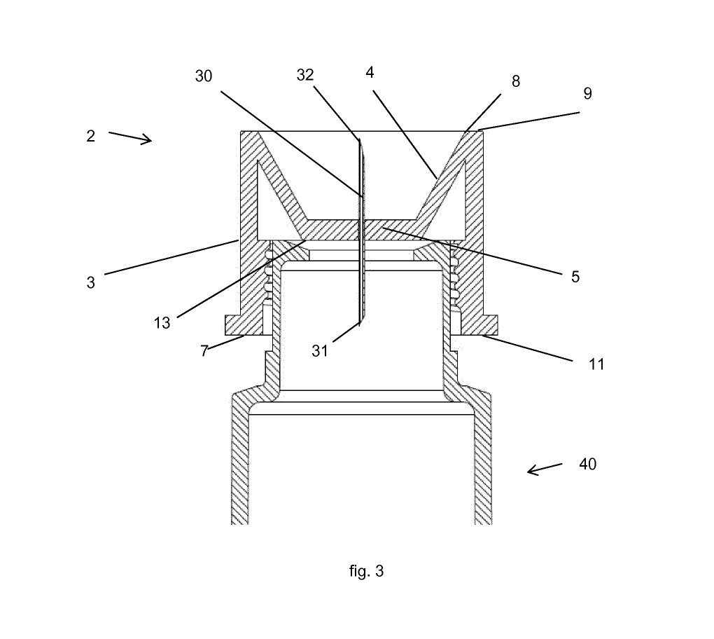

FIG. 3 shows the needle unit 2 being mounted to the needle mount of a drug delivery device 40. The needle unit is not fully mounted, and is shown in a retracted mode. In the retracted mode, the distal end 32 of the needle cannula 30 is in a position, which is proximal to the distal end 8 of the skirt 3, and whereby the needle cannula 30 may be safely accommodated within the skirt 3. As shown on the figure, the cannula tip 32 is shielded by the skirt 3, which thereby minimizes the risk of needle injury during the mounting process.

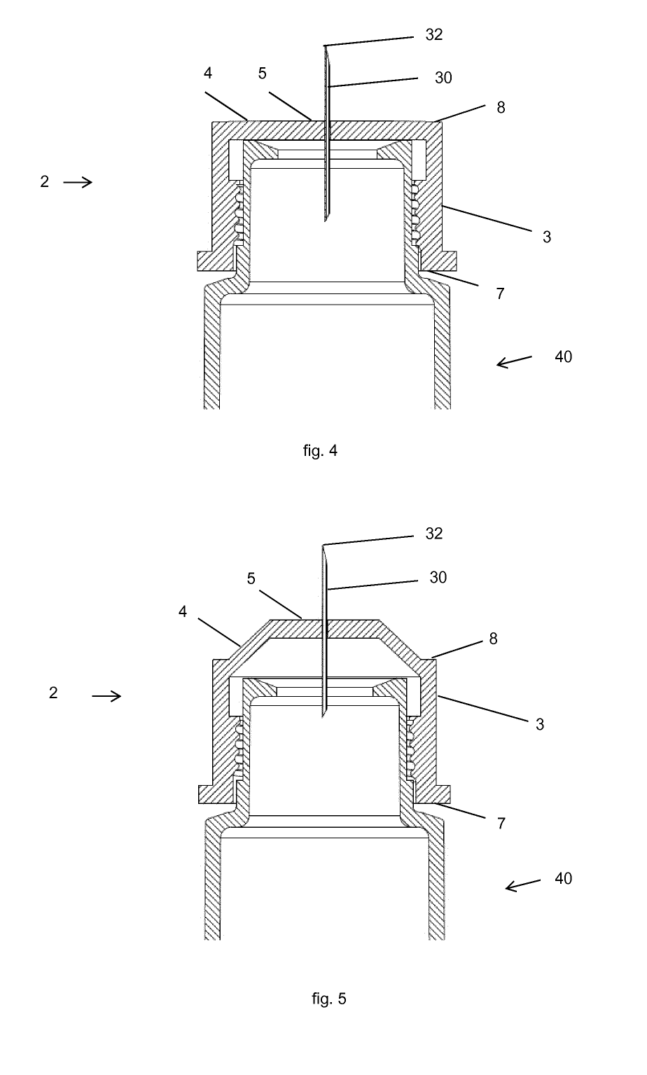

FIG. 4 shows an embodiment of the needle unit 2 being fully mounted on the needle mount, and is shown in an ejected mode. In the ejected mode, the distal end 32 of the needle 30 is in a position which is distal to the distal end 8 of the skirt 3, and whereby the needle cannula 30 may be ready for insertion. In the embodiment shown on FIG. 4 the transition portion 4 is compressible and thereby comprises a biasing member. On the figure, the skirt 3, the transition portion 4 and the central hub portion 5 is shown as an integrated unit which could be fabricated by injection moulding. However, the skirt 3, the transition portion 4 and the central portion 5 may also be separate components connected to each other. The transition portion 4 can be made of a compressible material or it can comprise a geometry providing the compressible or flexible properties. A geometry providing a compressible or flexible geometry could, e.g., be a corrugated structure, a spring or spiral structure. A compressible material could be a polymer.

The needle unit 2 can be switched between the retracted and the distal mode, as the transition portion 4 can deform. During deformation of the transition portion 4, the central hub portion 5 is moved in a longitudinal direction, as the two portions are connected. For embodiments comprising a biasing element, the biasing element urges the central portion in a proximal direction, and the biasing element may therefore result in a restoring effect, where the needle unit is urged back to the retracted mode.

FIG. 4 shows an embodiment of the needle assembly according to the present invention with the needle unit in an ejected mode. The transition portion 4 comprises a bi-stable element or member, and the needle unit 2 is therefore only stable in the retracted and the ejected mode. Any intermediate mode, between the retracted and the ejected mode, is unstable and may flip to one of the stable modes. The needle unit in the different modes may be described as if the transition portion 4 is having different shapes. In the retracted mode, the transition portion 4 in connection with hub portion 5 may be described as an inverted frustoconical shape, i.e, the shell of an inverted cone without a tip portion. In the ejected mode, the two portions 4 and 5 may be described as a frustoconical shape. Therefore, the needle unit switching from a retracted to an ejected mode, switches from an inverted frustoconical to a frustoconical shape. However, the shape is not necessarily inverted frustoconical as shown on FIG. 4, but may also be an inverted dome or any other shape providing a bi-stable structure, which ensures a stable alignment of the cannula along the central axis A.

FIG. 2 also shows a first 20 and a second sealing member 21 supported by a first 9 and a second sealing support portion 10, respectively. Thereby, in the retracted mode, the sealing elements seals off the distal and the proximal end of the needle assembly. However, the needle assembly may also be sealed off by a single sealing element being sufficiently long to cover both ends. The sealing support portion may comprise a flange 11 to increase the surface for supporting a seal. The figure shows, a flange 11 at the proximal end of the skirt, but a flange may also be provided at the distal end. The sealing elements may be a foil made of paper, aluminum and the like, and the sealing elements may be adapted to be automatically broken during the mounting process. The sealing elements could also be peelable foils removed before the needle unit is mounted on the drug delivery device. FIG. 2 further shows a close up of a window B around the needle tip and the first sealing member. In order to avoid unintentional penetration of the sealing member, there has to be a certain minimum distance d between the distal end 32 of the cannula 30 and the first sealing member 20.

One way of handling the needle assembly could be to remove the second seal covering the proximal end of the needle, or to mount the needle unit on the needle mount, whereby the seal is broken or in some other way is put in a state, where it allows the needle unit to be mounted, and allows the proximal end of the needle cannula 30 to penetrate a septum in a drug reservoir of the drug delivery device. At some point during the mounting process, a structure or a surface of the drug delivery device may interface or abut with a proximal surface 13 of the central hub portion 5. When the drug delivery device is further advanced relative to the needle unit, the structure of the drug delivery device will exert a force onto the surface 13, and the central hub portion 5 may be driven in a proximal direction.

If the transition portion 4 comprises a biasing member, the central hub portion will move continuously with the advancement of the drug delivery device until it has reached the ejected mode.

If the transition portion 4 comprises a bi-stable member, the central hub portion 5 may flip to the ejected mode, as soon as the force is exerted on the proximal surface, or the needle unit may be designed in such a way, that a threshold has to be exceeded before the needle unit flips or switches to the ejected mode. In this case, the central hub portion 5 may follow the drug delivery device until a certain advancement, which corresponds to the threshold. A further advancement will result in the needle hub switching to the ejected mode. For embodiments comprising a bi-stable element, the drug delivery device may be advanced until the device supports the central hub portion 5 in the ejected state, and thereby allows the cannula to be inserted without risking a retraction.

When the needle unit is dismounted, the process is reversed and the needle unit may switch from the ejected to the retracted mode. For embodiments comprising a bi-stable element, the retraction may occur automatically, or the retraction can be caused by a separate actuation like squeezing the needle unit, or in some other way exerting a small force to a distal surface of the central hub portion 5. In some embodiments, the proximal surface 13 of the central hub portion 5 may be provided with structures gripping a corresponding structure of the drug delivery device, and thereby allows the drug delivery device to exert a pull force on the central hub portion. For embodiments comprising a biasing member, the central hub portion may continuously follow the drug delivery device upon retraction.

Some preferred embodiments have been shown in the foregoing, but it should be stressed that the invention is not limited to these, but may be embodied in other ways within the subject matter defined in the following claims.

1. A needle assembly comprising a needle unit, where the needle unit comprises:
a hub with a skirt having a longitudinal central axis, where the skirt is having a proximal and a distal end, and an internal surface adapted to interface with a mounting portion of a drug delivery device;
a needle cannula having a proximal and a distal end;
a central hub portion holding the needle cannula;
a transition portion attached to the skirt portion, and wherein the transition portion connects the skirt with the central hub portion;
where the needle unit is adapted to be switched between:
a retracted mode, where the distal end the needle cannula is in a position which is proximal to the distal end of the skirt, and whereby the needle cannula may be safely accommodated within the skirt; and
an ejected mode, where the distal end of the needle cannula is in a position which is distal to the distal end of the skirt, and whereby the needle cannula may be ready for insertion; and
wherein the transition portion is adapted to deform, and thereby enable the switching between the modes, by allowing the central hub portion to be moved in a longitudinal direction.

2. A needle assembly according to claim 1 wherein the transition portion is adapted to change between a first shape in the retracted mode, and a second shape in the ejected mode.

3. A needle assembly according to claim 1
wherein the transition portion is adapted to position the central hub portion in a first position, when the needle unit is in the retracted mode; and
wherein the transition portion is adapted to position the central hub portion in a second position, when the needle unit is in the ejected mode; and
wherein the first position is proximal to the second position.

4. A needle assembly according to claim 1 further comprising:
a first sealing member; and wherein
the skirt is having a first sealing support portion at the distal end of the skirt, wherein said first sealing support portion is adapted to support the first sealing element.

5. A needle assembly according to claim 1 where the needle unit is adapted to be reversibly switched between the retracted and the ejected mode.

6. A needle assembly according to claim 1 wherein the needle unit is adapted to be switched between:
an initial retracted mode, wherein the needle assembly is in the retracted mode, the first sealing element is furthermore supported by the first sealing support portion, and the distal end of the needle cannula and the first sealing element is furthermore separated by a minimum distance; and
the ejected mode.

7. A needle assembly according to claim 4 wherein the first sealing element is adapted to be broken by the needle cannula, when the needle unit is switched between the initial retracted mode and the ejected mode.

8. A needle assembly according to claim 1 further comprising a second sealing support portion at the proximal end of the skirt, a second sealing element, and wherein the second sealing support portion is adapted to support the second sealing element.

9. A needle assembly according to claim 8 wherein the second sealing element is adapted to be broken by the drug delivery device, when the needle unit is mounted on the drug delivery device.

10. A needle assembly according to claim 1 wherein the internal surface of the skirt comprises attachment structure adapted to attach the needle unit to the needle mount of the injection device.

11. A needle assembly according to claim 1 wherein the needle unit is adapted to be switched between the retracted and the ejected mode, when the needle unit is mounted on the drug delivery device.

12. A needle assembly according to claim 1 wherein the central hub portion is having a proximal surface adapted to interface with a structure of the drug delivery device, and wherein the needle unit is adapted to be switched between the retracted and the ejected mode, when a force having a longitudinal component is exerted on the proximal surface, wherein the central hub portion may be driven in a longitudinal direction during mounting of the drug delivery device.

13. A needle assembly according to claim 1 wherein the transition portion comprises a biasing member exerting a force on the central hub portion to enable an automatic retraction from the ejected mode, when the drug delivery device is dismounted.

14. A needle assembly according to claim 1 wherein the transition portion comprises a bi-stable element, whereby the needle unit is adapted to be mechanically stable in the retracted and the ejected mode, and wherein the central hub portion, during the process of mounting the drug delivery device to the needle unit, may be switched between the retracted and the ejected mode through an intermediate mode, where the intermediate mode defines an unstable mode, where the central hub portion can flip to either of the two stable modes.

15. A method of handling a needle assembly according to claim 1 comprising:
providing the needle assembly; and
mounting the needle unit on the drug delivery device and thereby switching the needle unit from the retracted to the ejected mode.
